(19) United States
(12) Patent Application Publication
(10) Pub. No.: US 2018/0369111 A1
(43) Pub. Date: Dec. 27, 2018

(54) COMPOSITION COMPRISING BAICALIN AT A HIGH CONCENTRATION

(71) Applicant: L'OREAL, Paris (FR)

(72) Inventors: Karinne LUCET-LEVANNIER, Rueil Malmaison (FR); Maud WILLIEN, Vitry Sur Seine (FR)

(21) Appl. No.: 16/064,101

(22) PCT Filed: Dec. 23, 2016
(86) PCT No.: PCT/EP2016/082637

§ 371 Date: Jun. 20, 2018

(30) Foreign Application Priority Data
Dec. 23, 2015 (FR) 1563282

(51) Int. Cl. A61K 8/60; A61K 8/67; A61K 8/06; A61Q 19/08

(52) U.S. Cl. CPC A61K 8/602; A61K 8/671; A61K 8/673; A61K 2800/522; A61K 8/678; A61K 8/06; A61Q 19/08; A61K 8/676

(57) ABSTRACT

Composition comprising baicalin at a high concentration The invention relates to a composition comprising at least one aqueous phase comprising baicalin, or an extract containing same, baicalin being solubilized and present at a mass concentration comprised between 0.25% and 10% with respect to the total mass of the composition, said composition having a p H of 5 to 6 and said composition being an emulsion. The invention also relates to a method for preparing such a composition and the uses of such a composition, particularly in cosmetics.

The present invention relates to a composition, and in particular a cosmetic composition, comprising a high concentration of baicalin, a method for preparing such a composition, along with the uses of this composition and of baicalin in such a composition.

Numerous cosmetic compositions comprising baicalin as an antioxidant, or an extract containing same, are known in the prior art. However, baicalin is very weakly soluble in aqueous medium. It is therefore comprised in these compositions at a mass concentration less than 0.2%, thus limiting the antioxidant effect thereof. As such, the patent application DE102009037900 A1 describes for example compositions comprising baicalin at a mass content less than or equal to 0.2%. Similarly, the U.S. Pat. No. 7,427,690 B2 describes a composition comprising baicalin at a mass concentration equal to 0.2%. Moreover, the patent application EP 2729117 A1 describes photoprotective compositions comprising baicalin at a mass concentration equal to 0.2%. American patent U.S. Pat. No. 9,107,853 B2 relates to a cosmetic composition comprising from 0.1 to 1% by mass of baicalin combined with a hydrotropic agent, such as nicotinamide. The presence of a hydrotropic agent makes it possible to increase the concentration of baicalin in a composition but therefore requires the presence of such an agent in the composition. These compositions have a pH less than 5, furthermore, hydrotropic agents are used at very high levels, in practice 5% and require an aqueous phase of at least 15%. Moreover, caffeine and vitamin B3 are molecules having an inherent biological activity which is not necessarily compatible with the products wherein Baicalin is to be formulated.

Baicalin is a polyphenol displaying an antioxidant activity. There is thus a need, for consumers, to provide cosmetic compositions comprising baicalin, or an extract containing same, at a high concentration and having a greater antioxidant efficacy than the cosmetic compositions known in the prior art comprising baicalin. However, the solubility of baicalin is very low in aqueous medium. It is therefore very difficult to incorporate in a composition, in particular a cosmetic composition, at a high mass concentration, in particular at a mass concentration greater than 0.2%. At a mass concentration greater than 0.2%, the appearance of the presence of crystallized baicalin is observed. The present inventors have identified that the solubility of baicalin was increased with the pH. However, at a pH greater than 6, baicalin is chemically unstable and is degraded, thus causing the destruction thereof.

Moreover, baicalin causes yellowing of the cosmetic compositions comprising same, in particular when the concentration thereof increases.

Consequently, a technical problem lies in obtaining a cosmetic composition comprising baicalin, or an extract containing same, at a mass concentration greater than or equal to 0.2%.

A further technical problem lies in obtaining a stable cosmetic composition comprising baicalin, or an extract containing same.

A further technical problem lies in obtaining a cosmetic composition that is both stable and comprising baicalin, or an extract containing same, at a mass concentration greater than or equal to 0.2%.

A further technical problem lies in the incorporation of baicalin, or an extract containing same, at a mass concentration greater than or equal to 0.2%, in a cosmetic composition while protecting the baicalin from degradation or a recrystallization phenomenon.

A further technical problem lies in obtaining a cosmetic composition comprising baicalin, or an extract containing same, at a mass concentration greater than or equal to 0.2% and consequently having a greater antioxidant activity than the cosmetic compositions known in the prior art comprising baicalin.

The aim of the invention is that of solving the technical problems cited above. In particular, a first aim consists of providing a cosmetic composition comprising baicalin, or an extract containing same, at a mass concentration greater than or equal to 0.2%.

A further aim consists of providing a stable cosmetic composition comprising baicalin, or an extract containing same.

A further aim consists of providing a cosmetic composition that is both stable and comprising baicalin, or an extract containing same, at a mass concentration greater than or equal to 0.2%. A further aim consists of providing a preparation method suitable for incorporating baicalin, or an extract containing same, at a mass concentration greater than or equal to 0.2% in a cosmetic composition, without degrading or recrystallizing same.

A further aim consists of providing a cosmetic composition comprising baicalin, or an extract containing same, at a mass concentration greater than or equal to 0.2% and consequently having a greater antioxidant activity than the cosmetic compositions known in the prior art comprising less than 0.2% of baicalin.

Finally, a further aim consists of providing a cosmetic composition comprising baicalin, or an extract containing same, at a mass concentration greater than or equal to 0.2%, the color whereof is stable over time.

The patent application WO 2014/059225 discloses compositions with a mass concentration of baicalin of 0.5%, based on the total mass of the compositions. However, such compositions need hydrotropic agents to solubilize baicalin and their pH is of 4.5.

The inventors surprisingly discovered that it was possible to incorporate baicalin, or an extract containing same, at a high mass concentration, i.e. greater than 0.2% in a composition, in particular cosmetic, in particular in the aqueous phase of such a composition. Surprisingly, baicalin is not degraded and does not recrystallize in spite of the very low solubility thereof. The cosmetic composition obtained is thus advantageously stable.

In particular, the inventors discovered that with a mass concentration of baicalin equal to or greater than 0.2%, preferably 0.25%, the color of the composition comprising baicalin is pale yellow to yellow and has a less pronounced orange tint than compositions having a pH outside the 5-6 range, which is preferable for cosmetic compositions. Preferably, the color of the compositions according to the invention has an h* value (hue angle) comprised between 110 and 135.

As such, the present invention relates to a composition, preferably a cosmetic composition comprising at least one aqueous phase comprising baicalin, or an extract containing same, baicalin being solubilized and present at a mass concentration greater than or equal to 0.2%, preferably strictly greater than 0.2% by mass with respect to the total mass of the composition, said composition having a pH of 5 to 6.

The present invention also relates to a method for preparing such a composition, characterized in that it comprises steps for:

a) Mixing baicalin with at least water in order to obtain a mixture A containing baicalin;

b) Increasing the pH of mixture A to a pH greater than 6 by adding a base; and

c) Lowering the pH to a pH ranging from 5 to less than 6 by adding an acid, and optionally adjusted with a base.

In particular, the method implemented to incorporate baicalin, or an extract containing same, at a high mass concentration, i.e. for example greater than 0.5% in the aqueous phase of a composition consists, in a first transitory step, of completely dissolving the baicalin in aqueous phase in a basic medium using a base to raise the pH to a value greater than 6. Under these conditions, it is possible to prepare aqueous solutions comprising up to 10% baicalin as a mass concentration, without using hydrotropic agents.

The present invention also relates to the use of a high concentration of baicalin as an antioxidant in a cosmetic composition.

Finally, the invention relates to the use of the composition according to the invention for reducing the pigmentation induced by long UVA rays and/or by exposure to DUV (UV from daily exposure or Daily UV). The DUV values are obtained for example using a ORIEL 1000 solar simulator lamp (Model SOL-UV-4: serial number 101) with a Model 69920 type power supply filtered with a WG320 2.0 mm optical filter. The exposure band is then 290-440 nm. The invention is detailed hereinafter with reference to the more general description of the invention.

Baicalin has the following formula:

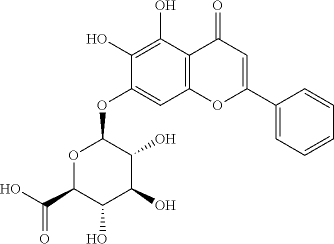 [SMILES: O=C(O)[C@H]1O[C@@H](Oc2cc3oc(-c4ccccc4)cc(=O)c3c(O)c2O)[C@H](O)[C@@H](O)[C@@H]1O]

Baicalin is a polyphenol comprising a carboxylic acid function. It is a structure consisting of a flavone and glucuronic acid. Glucuronic acid is not sufficiently polar to dissolve baicalin in aqueous medium at a high mass concentration, particularly greater than or equal to 0.2%, 0.3%, 0.4%, or 0.5%.

Baicalin is presented in the form of a yellow powder, having a purity optionally greater than 90 or 95%. Baicalin crystals may be presented in the form of needles. It is very weakly soluble, in particular in water at spontaneous pH.

Baicalin is available from MMP or Guilin Layn Natural Ingredients Corp. for example. Such an organic compound may be obtained as described in the patent application US2004/0067894 relating to the synthesis of tetrahydroxyflavones wherein the general formula comprises baicalin.

Baicalin may be used in the form of an extract of plant origin. Baicalin is a polyphenol (flavone) particularly extracted from skull cap root (scutellaria and particularly scutellaria baicalensis), based on traditional Chinese medicine. Baicalin has a very broad antioxidant activity profile in vitro and in vivo.

Preferentially, baicalin is comprised in the cosmetic composition in the completely dissolved state. No baicalin crystals remain, visible to the naked eye, with a microscope in polarized light or using any technique known to those skilled in the art.

According to one embodiment, the composition according to the invention is a composition, preferably a cosmetic composition, comprising at least baicalin, at least one base; and optionally at least one acid.

According to one embodiment, the composition according to the invention is a composition, preferably a cosmetic composition, comprising at least one aqueous phase comprising baicalin, or an extract containing same, solubilized, at a mass concentration greater than or equal to 0.5%, preferentially greater than or equal to 1% by mass and more preferentially greater than or equal to 1.5% by mass with respect to the total mass of the composition.

According to one embodiment, the composition according to the invention is a composition, preferably a cosmetic composition, comprising:
at least one aqueous phase comprising baicalin, or an extract containing same, solubilized, at a mass concentration greater than or equal to 0.2%, preferably strictly greater than 0.2%, or 0.25%, advantageously 0.3%, preferentially 0.35% for example greater than or equal to 0.4%, for example greater than or equal to 0.45%, and further for example greater than or equal to 0.5% by mass with respect to the total mass of the composition;
optionally at least one base and/or at least one acid.

According to one embodiment, the composition according to the invention is a composition comprising at least one aqueous phase comprising baicalin, or an extract containing same, solubilized, at a mass concentration ranging between 0.2% and 10%, for example from 0.2% to 8%, for example from 0.2% to 6% and further for example from 0.2% to 5%.

According to one embodiment, the composition according to the invention is a composition comprising at least one aqueous phase comprising baicalin, or an extract containing same, solubilized, at a mass concentration ranging between 0.2% and 10%, for example between 0.5% and 10%, for example from 0.75% to 8%, for example from 1% to 6% and more preferentially from 1.5% to 5%.

According to one embodiment, the composition according to the invention is a composition comprising at least one aqueous phase comprising baicalin, or an extract containing same, solubilized, at a mass concentration ranging between 0.25% and 10%, for example between 0.25% to 8%, for example from 0.25% to 6% and more preferentially from 0.25% to 5%.

The composition, preferably the composition according to the invention, comprises an aqueous phase and a hydrophobic phase.

According to one embodiment, the composition according to the present invention comprises, as a mass concentration, at least 20% water, advantageously at least 25% water, preferably at least 40% water with respect to the total mass of the composition.

According to one embodiment, the composition according to the present invention is an emulsion.

According to one alternative embodiment, the composition according to the invention is a single emulsion.

According to one alternative embodiment, the composition according to the invention is a multiple emulsion. It may consist of a triple emulsion.

According to one alternative embodiment, the composition according to the invention is a direct single emulsion. As such, according to one embodiment, the composition according to the present invention is an oil-in-water (O/W) emulsion.

According to one alternative embodiment, the composition according to the invention is an inverse single emulsion. As such, according to one embodiment, the composition according to the present invention is a water-in-oil (W/O) emulsion.

According to one alternative embodiment, the composition according to the invention is an aqueous solution.

Advantageously, the pH of the composition is between 5.1 and 5.9, and preferably between 5.1 and 5.6.

According to one alternative embodiment, the cosmetic composition according to the invention comprises an acid and a base.

According to one embodiment, the composition does not comprise a phosphate buffer.

According to one alternative embodiment, the composition according to the invention comprises at least one base. The base is particularly used to increase the pH of the initial aqueous solution comprising baicalin and consequently solubilize same. It can also be used to adjust the final pH of the composition between 5 and 6. The basic quantity is thus that sufficient to completely solubilize baicalin.

The base may be chosen from mineral bases such as for example alkaline metal hydroxides, sodium hydroxide, potassium hydroxide, ammonium hydroxides, ammonia, organic bases such as for example monoethanolamine, diethanolamine, triethanolamine, triisopropylamine, tri[(2-hydroxy) 1-propyl)] amine, N,N-dimethyl ethanolamine, 2-amino 2-methyl 1-propanol, 2-amino 2-methyl 1,3-propanediol, triethylamine, dimethylaminopropylamine and amphoteric bases (i.e. bases having both anionic and cationic functional groups) such as primary, secondary, tertiary or cyclic organic amines, amino acids. By way of example of amphoteric bases, mention may be made of glycine, lysine, arginine, taurine, histidine, alanine, valine, cysteine, trihydroxymrehylaminomethane (TRISTA), triethanolamine and any of the mixtures thereof.

According to one particular embodiment, the base of the composition is chosen from sodium hydroxide, potassium hydroxide, ammonium hydroxides, ammonia, monoethanolamine, diethanolamine, triethanolamine, tromethamine and any of the mixtures thereof.

According to one particular embodiment, the base of the composition is sodium hydroxide.

According to one particular embodiment, the base of the composition according to the invention is present at a mass concentration less than 0.5%, or less than 0.25% by mass with respect to the total mass of the composition.

According to one alternative embodiment, the composition according to the invention comprises at least one acid. It may be used to adjust the final pH of the composition between 5 and 6, preferably from 5.1 to 5.9.

The acid may be chosen from mineral acids such as hydrochloric acid, sulfuric acid, nitric acid, organic acids such as acetic acid, lactic acid, glycolic acid, mandelic acid, citric acid, ascorbic acid and any of the mixtures thereof.

The acid may be chosen from organic acids, such as stearic acid, palmitic acid, myristic acid, hydrophobic chain acrylate polymers such as for example pemulen TR2, anionic aqueous phase gelling agents such as for example acrylamido propane sulfonic acid homopolymers, and any of the mixtures thereof.

Advantageously, the acid may also be chosen from carboxylic or sulfonic acid polymers and any of the mixtures thereof.

Among the acid polymers, mention may be made of optionally modified carboxyvinyl polymers, such as the products marketed under the trade names Carbopol (CTFA name: carbomer) and Pemulen (CTFA name: Acrylates/C 10-30 akyl acrylate crosspolymer) by Goodrich; 2-acrylamido 2-methylpropane sulfonic acid polymers and copolymers, optionally cross-linked and/or neutralized, such as poly(2-acrylamido-2-methylpropanesulfonic acid) marketed by Hoechst under the trade name “Hostacerin AMPS” (CTFA name: Ammonium Polyacryldimethyltauramide); polysaccharide biopolymers such as xanthan gum, alginates, modified celluloses having at least one COOH group, such as for example carboxyalkylcelluloses; and mixtures thereof.

According to one particular embodiment, the acid of the composition according to the invention is chosen from hydrochloric acid, acetic acid, citric acid and any of the mixtures thereof.

According to one particular embodiment, the acid of the composition according to the invention is citric acid.

According to one particular embodiment, the acid of the composition according to the invention is chosen from anionic aqueous phase gelling agents such as for example acrylamido propane sulfonic acid homopolymers (particularly Hostacerine AMPS® having the INCI name AMMONIUM POLYACRYLOYLDIMETHYL TAURATE).

According to one particular embodiment, the acid of the composition according to the invention is present at a mass concentration less than 0.5%, or less than 0.25% by mass with respect to the total mass of the composition.

According to one particular embodiment, the composition according to the invention further comprises cosmetically acceptable agents and/or excipients.

The term “cosmetically acceptable” means compatible with the skin and/or integuments thereof, having a pleasant color, odor and texture and not giving rise to unacceptable discomfort (tingling, tightness, redness), liable to dissuade the consumer from using the composition.

According to one embodiment, the composition according to the invention further comprises at least one vitamin chosen from vitamin A (retinol), vitamin E (tocopherol), vitamin C (ascorbic acid), vitamin CG (ascorbyl glucoside), vitamin B5 (panthenol), derivatives of these vitamins (particularly esters) and any of the mixtures thereof.

According to one particular embodiment, the composition according to the invention further comprises vitamin E (tocopherol).

According to one particular embodiment, the composition according to the invention further comprises vitamin E (tocopherol) and/or vitamin CG (ascorbyl glucoside).

According to one particular embodiment, the composition according to the invention further comprises vitamin E (tocopherol) at a mass concentration between 0 and 1.5%.

According to one particular embodiment, the composition according to the invention further comprises vitamin E (tocopherol) at a mass concentration between 0.5 and 1.5% and vitamin CG (ascorbyl glucoside).

Preferably, the mass concentrations of vitamin range from 0.1% to 5%, and preferably from 0.2 to 2.5%, by mass with respect to the total mass of said composition.

According to one embodiment, the composition according to the invention may further comprise at least one cosmetically active ingredient (or “active agent”).

The active agent may be chosen from enzymes; antioxidants; flavonoids; moisturizing agents; anti-inflammatories; procyanidolic oligomers; depigmenting agents; alpha-hydroxyacids; retinoids; hydrocortisone; melatonin; extracts of algae, fungi, plants, yeasts, bacteria; steroids; matting agents such as fibers; tightening agents; ceramides; essential oils; and mixtures thereof; and any suitable agent for the end purpose of the composition. According to one advantageous embodiment, the cosmetic composition according to the invention may further comprise at least one agent chosen from vitamin CG, vitamin E, ellagic acid, resveratol; phenylethyl resorcinol; carnosine and any of the mixtures thereof.

The concentration of agents in the composition according to the invention may generally vary from 0.0001 to 10% by mass.

According to one embodiment, the mass concentrations of agents range from 0.01% to 5%, and preferably from 0.1 to 3%, by mass with respect to the total mass of said composition.

According to one embodiment, the composition according to the invention may further comprise at least one filler.

According to one alternative embodiment, the filler is an inorganic (mineral) filler.

According to one alternative embodiment, the filler is an organic filler.

According to one alternative embodiment, the fillers present in the composition are a mixture of at least one inorganic (mineral) filler and at least one organic filler.

The filler may be chosen from pigments, titanium oxide, red iron oxide, yellow iron oxide, black iron oxide, boron nitride, nacres, synthetic or natural mica, nacres comprising mica and titanium oxide, silica powder; talc; polyamide particles, particularly those sold under the trade name ORGASOL by Atochem; polyethylene powders; microspheres based on acrylic copolymers, such as those made of ethylene glycol dimethacrylate/lauryl methacrylate copolymer sold by Dow Corning under the trade name POLYTRAP;

expanded powders such as hollow microspheres and in particular, the microspheres sold under the trade name EXPANCEL by Kemanord Plast or under the trade name MICROPEARL F 80 ED by Matsumoto; silicone resin microbeads such as those sold under the trade name TOSPEARL by Toshiba Silicone; and mixtures thereof. These fillers may be present in quantities ranging from 0 to 20% by mass and preferably from 1 to 10% by mass with respect to the total mass of the composition.

According to one advantageous embodiment, the composition according to the invention further comprises at least one filler chosen from titanium oxide, red iron oxide, yellow iron oxide, black iron oxide, boron nitride, nacres, synthetic or natural mica, nacres comprising mica and titanium oxide and any of the mixtures thereof.

Advantageously, the presence of one or a plurality of fillers, in particular titanium oxide, boron nitride or nacres comprising mica and titanium oxide makes it possible to attenuate the color of the compositions according to the invention.

Preferably, the mass concentrations of filler range from 0.1% to 30%, and preferably from 0.5 to 15%, by mass with respect to the total mass of said composition.

According to one advantageous embodiment, the cosmetic composition according to the invention may further comprises at least one organic and/or mineral UV filter (filters of UV rays from sunlight).

The UV filters is a UV filter routinely used in cosmetics. It may be chosen in the positive list contained in Annex VI of the Regulation (EC) No 1223/2009, which specifies the list of UV filters authorized in cosmetics.

The UV filters of the composition according to the invention can be of different natures.

They can be organic, lipophilic, hydrophilic or insoluble.

The term “lipophilic UV filter” refers to any cosmetic or dermatological filter liable to be completely dissolved in the molecular state in a liquid fat phase or be solubilized in colloidal form (for example in micellar form) in a liquid fat phase.

The term “hydrophilic UV filter” refers to any cosmetic or dermatological filter liable to be completely dissolved in the molecular state in a liquid aqueous phase or be solubilized in colloidal form (for example in micellar form) in a liquid aqueous phase.

The term “insoluble UV filter” refers to any cosmetic or dermatological filter which is not defined as a lipophilic UV filter or as a hydrophilic UV filter, in particle form in liquid aqueous or fat phase.

The UV filters of the composition according to the invention can provide a UVA and/or UVB photoprotection.

According to one embodiment, the composition further comprises at least one UV filter chosen from organic hydrophilic UV filters, organic lipophilic UV filters, insoluble organic UV filters, mineral filters or any of the mixtures thereof.

According to one embodiment, the composition may comprise one or several bis-resorcinyl triazine derivatives as described and prepared according to the syntheses indicated in patent applications EP-A-0775 698.

Examples of such compounds suitable for use include:
2,4-bis {[4-(2-ethyl-hexyloxy)-2-hydroxy]-phenyl}-6-(4-methoxy-phenyl)-1,3,5-triazine;
2,4-bis{[4-(3-(2-propyloxy)-2-hydroxy-propyloxy)-2-hydroxy]-phenyl}-6-(4-methoxyphenyl)-1,3,5-triazine;
2,4-bis {[4-(2-ethyl-hexyloxy)-2-hydroxy]-phenyl}-6-[4-(2-methoxyethyl-carboxyl)-phenylamino]-1,3,5-triazine;
2,4-bis {[4-tris(trimethylsiloxy-silylpropyloxy)-2-hydroxy]-phenyl}-6-(4-methoxyphenyl)-1,3,5-triazine;
2,4-bis {[4-(2″-methylpropenyloxy)-2-hydroxy]-phenyl}-6-(4-methoxyphenyl)-1,3,5-triazine;
2,4-bis {[4-(1 ‘,1’,1′,3′,5′,5′,5′-heptamethyltrisiloxy-2″-methylpropyloxy)-2-hydroxy]-phenyl}-6-(4-methoxyphenyl)-1,3,5-triazine.
2,4-bis{[4-(3-(2-propyloxy)-2-hydroxy-propyloxy]-2-hydroxy]-phenyl}-6-[(4-ethylcarboxyl)-phenylamino]-1,3,5-triazine;
2,4-bis {[4-(2-ethyl-hexyloxy)-2-hydroxy]-phenyl}-6-(1-methylpyrrol-2-yl)-1,3,5-triazine.

More specifically, at least the compound 2,4-bis {[4-(2-ethyl-hexyloxy)-2-hydroxy]-phenyl}-6-(4-methoxy-phenyl)-1,3,5-triazine or Bis-Ethylhexyloxyphenol Methoxyphenyl Triazine (INCI name) such as the product sold under the trade name “TINOSORB S” by CIBA GEIGY.

According to one embodiment, the composition may comprise one or several dibenzoylmethane derivatives. Particularly mention may be made of, but is not limited to:
2-methyldibenzoylmethane,
4-methyldibenzoylmethane,
4-isopropyldibenzoylmethane,
4-tert.-butyldibenzoylmethane,
2,4-dimethyldibenzoylmethane,
2,5-dimethyldibenzoylmethane,
4,4′-diisopropyldibenzoylmethane,
4,4′-dimethoxydibenzoylmethane,
4-tert.-butyl-4′-methoxydibenzoylmethane,
2-methyl-5-isopropyl-4′-methoxydibenzoylmethane,
2-methyl-5-tert-butyl-4′-methoxydibenzoylmethane,
2,4-dimethyl-4′-methoxydibenzoylmethane,
2,6-dimethyl-4-tert-butyl-4′-methoxydibenzoylmethane.

According to one embodiment, the composition may comprise one or several benzylidene camphor derivatives. Mention can be made in particular of:

3-Benzylidene camphor manufactured under the name “MEXORYL SD” by CHIMEX,

4-Methylbenzylidene camphor sold under the name “EUSOLEX 6300” by MERCK,

Benzylidene Camphor Sulfonic Acid manufactured under the name “MEXORYL SL” by CHIMEX,

Camphor Benzalkonium Methosulfate manufactured under the name “MEXORYL SO” by CHIMEX,

Polyacrylamidomethyl Benzylidene Camphor manufactured under the name “MEXORYL SW” by CHIMEX.

Terephthalylidene Dicamphor Sulfonic Acid marketed under the name MEXORYL SX by CHIMEX.

The organic UV filters can also be selected from anthranilics; cinnamic derivatives; salicylic derivatives; benzophenone derivatives; phenyl benzotriazole derivatives; benzalmalonate derivatives particularly those cited in the U.S. Pat. No. 5,624,663; phenyl benzimidazole derivatives; imidazolines; 4,4-diarylbutadiene derivatives; bis-benzoazolyl derivatives as described in the patents EP669323 and U.S. Pat. No. 2,463,264; p-aminobenzoic acid (PABA) derivatives; methylene bis-(hydroxyphenyl benzotriazole) derivatives as described in the applications U.S. Pat. No. 5,237,071, U.S. Pat. No. 5,166,355, GB2303549, DE 197 26 184 and EP893119; benzoxazole derivatives as described in the patent applications EP0832642, EP1027883, EP1300137 and DE10162844; filter polymers and filter silicones such as those particularly described in the application WO-93/04665; dimers derived from -alkylstyrene such as those described in the patent application DE19855649; 4,4-diarylbutadienes as described in the applications EP0967200, DE19746654, DE19755649, EP-A-1008586, EP1133980 and EP133981; other merocyanine derivatives such as those described in WO04006878, WO05058269 and WO06032741 and the mixtures thereof.

Examples of organic UV filters used in the composition according to the invention, include those referred to hereinafter using the INCI name thereof:

Lipophilic UV-A Filters:

### Dibenzoylmethane Derivatives:

Isopropyl Dibenzoylmethane; Butyl Methoxydibenzoylmethane

### Aminobenzophenones:

n-hexyl 2-(4-diethylamino-2-hydroxybenzoyl)-benzoate particularly sold under the trade name “UVINUL A+” by BASF;
1,1′-(1,4-piperazinediyl)bis[1-[2-[4-(diethylamino)-2-hydroxybenzoyl]phenyl]-methanone (CAS 919803-06-8)

According to a preferred embodiment the composition according to the invention comprises the UV filter of INCI name diethylamino hydroxybenzoyl hexyl benzoate (sold for example under the trade name “UVINUL A+”)

### Anthranilic Derivatives:

Menthyl anthranilate particularly sold under the trade name “NEO HELIOPAN MA” by SYMRISE;
   4,4-diarylbutadiene Derivatives:
1,1-dicarboxy (2,2′-dimethyl-propyl)-4,4-diphenylbutadiene;

### Merocyanine Derivatives:

Octyl-5-N,N-diethylamino-2-phenysulfonyl-2,4-pentadienoate;

According to one particular embodiment, the composition according to the invention comprises Butyl Methoxydibenzoylmethane (also known under the name Avobenzone).

Hydrophilic UV-A Filters:

The bis-benzoazolyl derivatives as described in the patents EP 669 323, and U.S. Pat. No. 2,463,264 and more specifically the compound Disodium Phenyl Dibenzimidazo tetra-sulfonate sold under the trade name “NEO HELIOPAN AP” by SYMRISE;

According to one particular embodiment, the composition according to the invention comprises Terephthalylidene Dicamphor Sulfonic Acid, (sold for example under the trade name Mexoryl SX).

Lipophilic UV-B Filters:

### Para-aminobenzoates:

### Ethyl PABA;

### Ethyl Dihydroxypropyl PABA;

Ethylhexyl Dimethyl PABA (ESCALOL 507 from ISP);

### Salicylic Derivatives:

Homosalate particularly sold under the name “Eusolex HMS” by Rona/EM Industries; Ethylhexyl Salicylate particularly sold under the name “NEO HELIOPAN OS” by SYMRISE;
Dipropyleneglycol Salicylate particularly sold under the name “DIPSAL” by SCHER; TEA Salicylate sold under the name “NEO HELIOPAN TS” by SYMRISE;

### Cinnamates

Ethylhexyl Methoxycinnamate particularly sold under the trade name “PARSOL MCX” by DSM Nutritional Products, Inc.;
Isopropyl Methoxy cinnamate;
Isoamyl Methoxy cinnamate particularly sold under the trade name “NEO HELIOPAN E 1000” by SYMRISE;

### Diisopropyl Methylcinnamate;

### Cinnoxate;

### Glyceryl Ethylhexanoate Dimethoxycinnamate;

β,β′-diphenylacrylate derivatives:
Etocrylene, particularly sold under the trade name “UVINUL N35” by BASF; Octocrylene, particularly sold under the trade name “UVINUL N539” by BASF;

### Benzylidene Camphor Derivatives:

3-Benzylidene camphor manufactured under the name “MEXORYL SD” by CHIMEX; Methylbenzylidene camphor sold under the name “EUSOLEX 6300” by MERCK;
Polyacrylamidomethyl Benzylidene Camphor manufactured under the name “MEXORYL SW” by CHIMEX;

### Triazine Derivatives:

Ethylhexyl triazone particularly sold under the trade name “UVINUL T150” by BASF;
Diethylhexyl Butamido Triazone particularly sold under the trade name “UVASORB HEB” by SIGMA 3V;
2,4,6-tris(dineopentyl 4′-amino benzalmalonate)-s-triazine;
2,4,6-tris(diisobutyl 4′-amino benzalmalonate)-s-triazine;
2,4-bis(dineopentyl 4′-amino benzalmalonate)-6-(n-butyl 4′-aminobenzoate)-s-triazine;
2,4-bis(n-butyl 4′-amino benzoate)-6-(aminopropyltrisiloxane)-s-triazine; the symmetrical triazine filters described in the U.S. Pat. No. 6,225,467, the application WO 2004/085412 (see compounds 6 and 9) or the document “Symmetrical Triazine Derivatives” IP.COM Journal, IP.COM INC. WEST HENRIETTA, N.Y., US (Sep. 20, 2004) particularly 2,4,6-tris-(biphenyl)-1,3,5-triazine (particularly 2,4,6-tris(biphenyl-4-yl-1,3,5-triazine) and 2,4,6-tris(terphenyl)-1,3,5-triazine, the latter two filters being described in the BEIERSDORF applications WO 06/035000, WO 06/034982, WO 06/034991, WO 06/035007, WO 2006/034992, WO 2006/034985).

### Imidazoline Derivatives:

### Ethylhexyl Dimethoxybenzylidene Dioxoimidazoline Propionate,

### Benzalmalonate Derivatives:

Polyorganosiloxanes with a benzalmalonate function such as Polysilicone-15 particularly sold under the trade name “PARSOL SLX” by DSM Nutritional Products, Inc.;
Di-neopentyl 4′-methoxybenzalmalonate;

According to one particular embodiment, the composition according to the invention comprises one or several UV filters chosen from the following filters: Ethylhexylsalicylate (sold in particular under the name “NEO HELIOPAN OS” by SYMRISE); Octocrylene (sold in particular under the trade name “UVINUL N539” by BASF); Ethylhexyl triazone (sold in particular under the trade name “UVINUL T150”) and any mixtures thereof.

Hydrophilic UV-B Filters:

The following p-aminobenzoic acid (PABA) derivatives:

### PABA,

### Glyceryl PABA and

PEG-25 PABA particularly sold under the trade name “UVINUL P25” by BASF.
Phenylbenzimidazole Sulfonic Acid particularly sold under the trade name “EUSOLEX 232” by MERCK,
ferulic acid,
salicylic acid,
DEA methoxycinnamate,
Benzylidene Camphor Sulfonic Acid manufactured under the name “MEXORYL SL” by CHIMEX,
Camphor Benzalkonium Methosulfate manufactured under the name “MEXORYL SO” by CHIMEX,

V/Combined Lipophilic UVA and UVB Filters:

### Benzophenone Derivatives

Benzophenone-1 particularly sold under the trade name “UVINUL 400” by BASF;
Benzophenone-2 particularly sold under the trade name “UVINUL D50” by BASF;
Benzophenone-3 or Oxybenzone particularly sold under the trade name “UVINUL M40” by BASF;
Benzophenone-6 particularly sold under the trade name “Helisorb 11” by Norquay;
Benzophenone-8 particularly sold under the trade name “Spectra-Sorb UV-24” by American Cyanamid;

### Benzophenone-10;

### Benzophenone-11;

### Benzophenone-12;

### Phenyl Benzotriazole Derivatives:

Drometrizole Trisiloxane particularly sold under the name “Silatrizole” by RHODIA CHIMIE or manufactured under the name “Meroxyl XL” by CHIMEX;
Methylene bis-Benzotriazolyl Tetramethylbutylphenol, sold in solid form particularly under the trade name “MIXXIM BB/100” by FAIRMOUNT CHEMICAL or in micronized form in aqueous dispersion particularly under the trade name “TINOSORB M” by CIBA SPECIALTY CHEMICALS;

According to one particular embodiment, the composition according to the invention comprises one or several UV filters chosen from the following filters: Drometrizole Trisiloxane (sold in particular under the name “Silatrizole” by RHODIA CHIMIE or manufactured under the name “Meroxyl XL” by CHIMEX), Methylene bis-Benzotriazolyl Tetramethylbutylphenol possibly in micronized form and any of the mixtures thereof.

Benzoxazole derivatives:
2,4-bis-[5-1(dimethylpropyl)benzoxazol-2-yl-(4-phenyl)-imino]-6-(2-ethylhexyl)-imino-1,3,5-triazine particularly sold under the name Uvasorb K2A by Sigma 3V.

### Combined Hydrophilic UVA and UVB Filters:

Benzophenone derivatives comprising at least one sulfonic radical such as
Benzophenone-4 particularly sold under the trade name “UVINUL MS 40” by BASF,

### Benzophenone-5 and

### Benzophenone-9.

### Merocyanine Family UV Filters

The cosmetic composition according to the invention may comprise one or a plurality of UV filters from the merocyanine family.

The merocyanine compounds comply with the following formula (1) along with the E/E- or E/Z-isomeric geometric forms thereof:
(1)

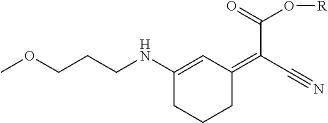

wherein
R is a C₁-C₂₂ alkyl group, a C₂-C₂₂ alkenyl group, a C₂-C₂₂ alkinyl group, a C₃-C₂₂ cycloalkyl group or a C₃-C₂₂ cycloalkenyl group, said groups optionally being interrupted by one or more O.

The merocyanine compounds according to the invention may be in the E/E-, E/Z-isomeric geometric forms thereof.

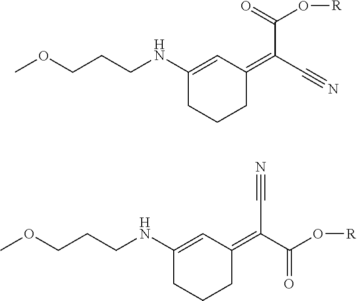

The preferential compounds according to formula (1) are those wherein:
R is a C₁-C₂₂ alkyl, optionally interrupted by one or more O.
Of the compounds according to formula (1), those chosen from the following compounds along with the E/E-, E/Z-isomeric geometric forms thereof will more particularly be used:

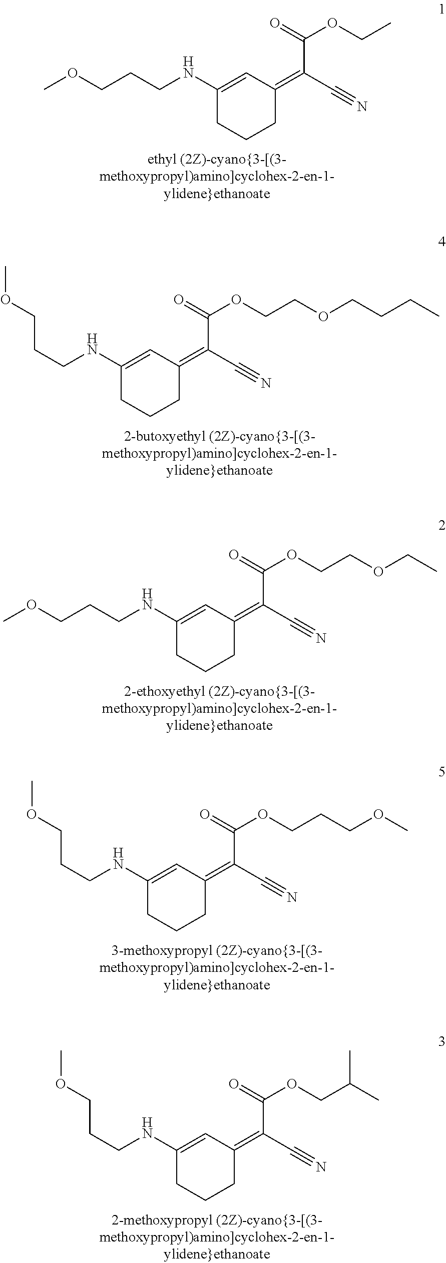

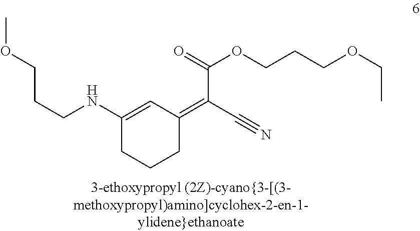 [SMILES: CCOCCCOC(=O)/C(C#N)=C1\C=C(NCCCOC)CCC1]

According to one embodiment of the invention, the compound 2-ethoxyethyl (2Z)-cyano{3-[(3-methoxypropyl)-amino]cyclohex-2-en-1-ylidene}ethanoate (2) will be used in the E/Z geometric configuration thereof having the following structure;

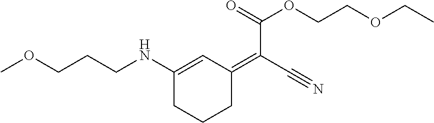 [SMILES: CCOCCOC(=O)/C(C#N)=C1\C=C(NCCCOC)CCC1]

and/or in the E/E geometric configuration thereof having the following structure:

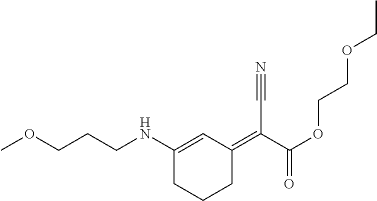 [SMILES: CCOCCOC(=O)/C(C#N)=C1/C=C(NCCCOC)CCC1]

The compounds according to formula (1) may be prepared according to the protocols described in Pat. Appl. WO 2007/071582, in IP.com Journal (2009), 9(5A), 29-30 IPCOM000182396D under the title “Process for producing 3-amino-2-cyclohexan-1-ylidene compounds” and in U.S. Pat. No. 4,749,643 on col, 13, line 66-col. 14, line 57 and the references cited in this respect.

According to one embodiment, the composition comprises one or several UV filters chosen from Terephthalylidene Dicamphor Sulfonic Acid, Butyl Methoxydibenzoylmethane, Drometrizole Trisiloxane, Octocrylene, Ethylhexylsalicylate, Methylene bis-Benzotriazolyl Tetramethylbutylphenol, Ethylhexyl triazone, Bis-Ethylhexyloxyphenol Methoxyphenyl Triazine, diethylamino hydroxybenzoyl hexyl benzoate and any of the mixtures thereof.

According to one embodiment, the composition comprises at least one organic UV filter chosen from among the following: Drometrizole Trisiloxane, Ethylhexyl triazone and diethylamino hydroxybenzoyl hexyl benzoate and any of the mixtures thereof. It can further comprise one or several filters of a non-organic nature, for example mineral.

According to one embodiment, the composition may comprise one or several lipophilic UV filters.

According to one embodiment, the quantity of the UV filter or filters, present in the composition according to the invention, may range from 0.001 to 50% by mass, with respect to the total mass of the composition. It ranges for example from 10 to 50% by mass, or for example from 15 to 50% by mass, and for example ranges from 20 to 40% by mass, with respect to the total mass of the composition. According to one particular embodiment, the concentration of UV filters in the composition according to the invention ranges from 1 to 50%, preferably from 1 to 30% by mass with respect to the total mass of the composition.

According to one embodiment, the Terephthalylidene Dicamphor Sulfonic Acid content of the composition according to the invention is from 3 to 12%, with respect to the total mass of the composition.

According to one embodiment, the Butyl Methoxydibenzoylmethane content of the composition according to the invention is from 1 to 5%, with respect to the total mass of the composition.

According to one embodiment, the Drometrizole Trisiloxane content of the composition according to the invention is from 1 to 4%, optionally from 2 to 3% with respect to the total mass of the composition.

According to one embodiment, the Octocrylene content of the composition according to the invention is from 0.5 to 2.5%, with respect to the total mass of the composition.

According to one embodiment, the Ethylhexylsalicylate content of the composition according to the invention is from 2 to 8%, with respect to the total mass of the composition.

According to one embodiment, the Methylene bis-Benzotriazolyl Tetramethylbutylphenol content of the composition according to the invention is from 1 to 4%, with respect to the mass of the composition.

According to one embodiment, the Ethylhexyl triazone content of the composition according to the invention is from 1 to 4%, optionally from 2 to 3%, with respect to the total mass of the composition.

According to one embodiment, the Bis-Ethylhexyloxyphenol Methoxyphenyl Triazine content of the composition according to the invention is from 0.5 to 4.5%, with respect to the total mass of the composition.

According to one embodiment, the diethylamino hydroxybenzoyl hexyl benzoate content of the composition according to the invention is from 0.5 to 5%, optionally from 1 to 4% with respect to the total mass of the composition.

The composition according to the invention may also comprise mineral UV filters which are generally pigments. The pigments may be coated or uncoated.

The coated pigments are pigments which have undergone one or a plurality of chemical, electronic, mechanochemical and/or mechanical surface treatments with compounds as described for example in Cosmetics & Toiletries, February 1990, Vol. 105, p. 53-64, such as amino acids, beeswax, fatty acids, fatty alcohols, anionic surfactants, lecithins, sodium, potassium, zinc, iron or aluminum salts of fatty acids, metal (titanium or aluminum) alkoxides, polyethylene, silicones, proteins (collagen, elastin), alkanolamines, silicon oxides, metal oxides or sodium hexametaphosphate.

According to the prior art, the silicones are organosilicate polymers or oligomers with a linear or cyclic, branched or cross-linked structure, with a variable molecular weight, obtained by polymerization and/or polycondensation of suitably functionalized silanes, and essentially consisting of a repetition of primary structural units wherein the silicon atoms are interconnected by oxygen atoms (siloxane bond), optionally substituted hydrocarbon radicals being directly bound via a carbon atom on said silicon atoms.

The term “silicones” also covers the silanes required for the preparation thereof, particularly, alkyl silanes.

The silicones used for coating pigments suitable for the present invention are preferably selected from the group containing alkyl silanes, polydialkylsiloxanes, and polyalkylhydrogen siloxanes. More preferentially, the silicones are selected from the group containing octyl trimethyl silane, polydimethylsiloxanes and polymethylhydrogen siloxanes.

Obviously, prior to the treatment thereof with silicone, the metal oxide pigments may have been treated with other surface agents, particularly cerium oxide, alumina, silica, aluminum compounds, silicon compounds, or mixtures thereof.

The coated pigments are, for example, titanium oxides coated with:
silica such as the product “SUNVEIL” from IKEDA and the product “Eusolex T-AVO” from MERCK
silica and iron oxide such as the product “SUNVEIL F” from IKEDA,
silica and alumina such as the products “MICROTITANIUM DIOXIDE MT 500 SA” and “MICROTITANIUM DIOXIDE MT 100 SA” from TAYCA, “TIOVEIL” from TIOXIDE, and “Mirasun TiW 60” from Rhodia,
alumina such as the products “TIPAQUE TTO-55 (B)” and “TIPAQUE TTO-55 (A)” from ISHIHARA, and “UVT 14/4” from KEMIRA,
alumina and aluminum stearate such as the product “MICROTITANIUM DIOXIDE MT 100 TV, MT 100 TX, MT 100 Z, MT-01” from TAYCA, the products “Solaveil CT-10 W”, “Solaveil CT 100” and “Solaveil CT 200” from UNIQEMA,
silica, alumina and alginic acid such as the product “MT-100 AQ” from TAYCA,
alumina and aluminum laurate such as the product “MICROTITANIUM DIOXIDE MT 100 S” from TAYCA,
iron oxide and iron stearate such as the product “MICROTITANIUM DIOXIDE MT 100 F” from TAYCA,
zinc oxide and zinc stearate such as the product “BR351” from TAYCA,
silica and alumina and treated with a silicone such as the products “MICROTITANIUM DIOXIDE MT 600 SAS”, “MICROTITANIUM DIOXIDE MT 500 SAS” or “MICROTITANIUM DIOXIDE MT 100 SAS” from TAYCA,
silica, alumina, aluminum stearate and treated with a silicone such as the product “STT-30-DS” from TITAN KOGYO,
silica and treated with a silicone such as the product “UV-TITAN X 195” from KEMIRA, or the product SMT-100 WRS from TAYCA.
alumina and treated with a silicone such as the products “TIPAQUE TTO-55 (S)” from ISHIHARA, or “UV TITAN M 262” from KEMIRA, triethanolamine such as the product “STT-65-S” from TITAN KOGYO,
stearic acid such as the product “TIPAQUE TTO-55 (C)” from ISHIHARA,
sodium hexametaphosphate such as the product “MICROTITANIUM DIOXIDE MT 150 W” from TAYCA.

Further titanium oxide pigments treated with a silicone are for example TiO2 treated with octyl trimethyl silane such as that sold under the trade name “T 805” by DEGUSSA SILICES, TiO2 treated with a polydimethylsiloxane such as that sold under the trade name “70250 Cardre UF TiO2SI3” by CARDRE, anatase/rutile TiO2 treated with a polydimethylhydrogen siloxane such as that sold under the trade name “MICRO TITANIUM DIOXIDE USP GRADE HYDROPHOBIC” by COLOR TECHNIQUES.

The uncoated titanium oxide pigments are for example sold by TAYCA under the trade names “MICROTITANIUM DIOXIDE MT 500 B” or “MICROTITANIUM DIOXIDE MT600 B”, by DEGUSSA under the trade name “P 25”, by WACKHER under the trade name “Oxyde de titane transparent PW”, by MIYOSHI KASEI under the trade name “UFTR”, by TOMEN under the trade name “ITS” and by TIOXIDE under the trade name “TIOVEIL AQ”.

The uncoated zinc oxide pigments are for example
those marketed under the trade name “Z-cote” by Sunsmart;
those marketed under the trade name “Nanox” by Elementis;
those marketed under the trade name “Nanogard WCD 2025” by Nanophase Technologies;

The coated zinc oxide pigments are for example
those marketed under the trade name “Z-COTE HP1” by SUNSMART (ZnO coated with dimethicone);
those marketed under the trade name “CS-5 zinc oxide” by Toshibi (ZnO coated with polymethylhydrogen siloxane);
those marketed under the trade name “Nanogard Zinc Oxide FN” by Nanophase Technologies (in 40% dispersion in Finsolv TN, C12-C15 alcohol benzoate);
those marketed under the trade name “DAITOPERSION ZN-30” and “DAITOPERSION Zn-50” by Daito (dispersions in cyclopolymethylsiloxane/oxyethylenated polydimethylsiloxane, containing 30% or 50% zinc oxides coated with silica and polymethylhydrogen siloxane);
those marketed under the trade name “NFD Ultrafine ZnO” by Daikin (ZnO coated with perfluoroalkyl phosphate and perfluoroalkylethyl-based copolymer in dispersion in cyclopentasiloxane);
those marketed under the trade name “SPD-Z1” by Shin-Etsu (ZnO coated with silicone-grafted acrylic polymer, dispersed in cyclodimethylsiloxane);
those marketed under the trade name “Escalol Z100” by ISP (ZnO treated with alumina and dispersed in the ethylhexyl methoxycinnamate/PVP-hexadecene copolymer/methicone mixture);
those marketed under the trade name “Fuji ZnO-SMS-10” by Fuji Pigment (ZnO coated with silica and polymethylsilsesquioxane);
those marketed under the trade name “Nanox Gel TN” by Elementis (ZnO in 55% dispersion in C12-C15 alcohol benzoate with hydroxystearic acid polycondensate). The uncoated cerium oxide pigments are sold for example under the trade name “COLLOIDAL CERIUM OXIDE”.

The uncoated iron oxide pigments are for example sold by ARNAUD under the trade names “NANOGARD WCD 2002 (FE 45B)”, “NANOGARD IRON FE 45 BL AQ”, “NANOGARD FE 45R AQ, “NANOGARD WCD 2006 (FE 45R)”, or by MITSUBISHI under the trade name “TY-220”.

The coated iron oxide pigments are for example sold by ARNAUD under the trade names “NANOGARD WCD 2008 (FE 45B FN)”, “NANOGARD WCD 2009 (FE 45B 556)”, “NANOGARD FE 45 BL 345”, “NANOGARD FE 45 BL”, or by BASF under the trade name “TRANSPARENT IRON OXIDE”.

It is also possible to cite metal oxide mixtures, particularly of titanium dioxide and cerium dioxide, including the mixture of equal weights of titanium dioxide and cerium dioxide coated with silica, sold by IKEDA under the trade name “SUNVEIL A”, and the mixture of titanium dioxide and zinc dioxide coated with alumina, silica and silicone such as the product “M 261” sold by KEMIRA or coated with alumina, silica and glycerin such as the product “M 211” sold by KEMIRA.

The pigments may be introduced into the compositions according to the invention as is or in pigment paste form, i.e. in a mixture with a dispersion agent, as described for example in the document GB-A-2206339.

According to one particular embodiment, the composition according to the invention further comprises an organic UV filter and a mineral UV filter:

According to one particular embodiment, the composition according to the invention further comprises an association of UV filters as described in the patent FR 2 977 490, the application WO 2013/004777 or the application US-2014-0134120.

According to one particular embodiment, the composition according to the invention further comprises a UV filter chosen from Butyl Methoxydibenzoylmethane, homosalate, octocrylene and ethyl hexyl salicylate.

According to one embodiment, the composition according to the invention may further comprise at least one emulsifier.

When the composition according to the invention is a water-in-oil (W/O) or oil-in-water (O/W) emulsion, the proportion of the oil phase of the emulsion may range from 5 to 80% by mass, and preferably from 5 to 50% by mass with respect to the total mass of the composition. The emulsions generally contain at least one emulsifier chosen from amphoteric, anionic, cationic or non-ionic emulsifiers, used alone or in a mixture, and optionally a co-emulsifier. The emulsifiers are suitably selected according to the emulsion to be obtained (W/O or O/W). The emulsifier and the co-emulsifier are generally present in the composition, in a proportion optionally ranging for example from 0.3% to 30% by mass, and preferably from 0.5% to 20% by mass with respect to the total mass of the composition.

For W/O emulsions, mention may be made for example as emulsifiers of dimethicone copolyols such as the mixture of cyclomethicone and dimethicone copolyol, sold under the trade name “DC 5225 C” by Dow Corning, and alkyl-dimethicone copolyols such as Laurylmethicone copolyol sold under the trade name “Dow Corning 5200 Formulation Aid” by Dow Corning; and Cetyl dimethicone copolyol sold under the trade name ABIL EM 90R by Goldschmidt, or the polyglycerol-4 isostearate/cetyl dimethicone copolyol/hexyl laurate mixture sold under the trade name ABIL WE 09 by Goldschmidt. One or a plurality of co-emulsifiers may also be added. Advantageously, the co-emulsifiers may be chosen in the group comprising alkylated polyol esters. By way of example of alkylated polyol esters, particular mention may be made of glycerol and/or sorbitan esters and for example polyglycerol isostearate, such as the product sold under the trade name Isolan GI 34 by Goldschmidt, sorbitan isostearate, such as the product sold under the trade name Arlacel 987 by ICI, sorbitan and glycerol isostearate, such as the product sold under the trade name Arlacel 986 by ICI, and any of the mixtures thereof.

It is also possible to use as a W/0 emulsion surfactant, a cross-linked solid elastomeric organopolysiloxane comprising at least one oxyalkylene group, such as those obtained according to the procedure in examples 3, 4 and 8 of the document U.S. Pat. No. 5,412,004 and the examples of the document U.S. Pat. No. 5,811,487, particularly the product of example 3 (example of synthesis) of the U.S. Pat. No. 5,412,004 and such as that marketed under the reference KSG 21 by Shin Etsu.

For O/W emulsions, mention may be made for example as emulsifiers, of non-ionic surfactants, and particularly saturated and unsaturated chain polyol and fatty acid esters comprising for example from 8 to 24 carbon atoms and preferably from 12 to 22 carbon atoms, and oxyalkylenated derivatives thereof, i.e. comprising oxyethylene and/or oxypropylene units, such as C8-C24 glyceryl and fatty acid esters, and oxyalkylenated derivatives thereof; C5-C24 polyethylene glycol and fatty acid esters, and oxyalkylenated derivatives thereof; C8-C24 sorbitan and fatty acid esters, and oxyalkylenated derivatives thereof; C8-C24 sugar (sucrose, glucose, alkyl glucose) and fatty acid esters, and oxyalkylenated derivatives thereof; fatty alcohol ethers; C8-C24 sugar and fatty alcohol ethers, and mixtures thereof.

By way of glyceryl and fatty acid ester, particular mention may be made of glyceryl stearate (glyceryl mono-, di- and/or tri-stearate) (CTFA name: glyceryl stearate) or glyceryl ricinoleate, and mixtures thereof.

By way of polyethylene glycol and fatty acid ester, particular mention may be made of polyethylene glycol stearate (polyethylene glycol mono-, di- and/or tri-stearate), and more specifically polyethylene glycol 50 EO monostearate (CTFA name: PEG-50 stearate), polyethylene glycol 100 EO monostearate (CTFA name: PEG-100 stearate and mixtures thereof.

Mixtures of these surfactants may also be used, such as for example the product containing Glyceryl stearate and PEG-100 stearate, marketed under the trade name ARLACEL 165 by Uniqema, and the product containing Glyceryl stearate (glyceryl mono-distearate) and potassium stearate, marketed under the trade name TEGIN by Goldschmidt (CTFA name: glyceryl stearate SE). By way of fatty acid and glucose or alkyl glucose ester, particular mention may be made of glucose palmitate, alkyl glucose sesquistearates such as methyl glucose sesquistearate, alkyl glucose palmitates such as methyl glucose or ethyl glucose palmitate, methyl glucoside fatty esters and more especially methyl glucose and oleic acid diester (CTFA name: Methyl glucose dioleate); the composite ester of methyl glucoside and the oleic acid/hydroxystearic acid mixture (CTFA name: Methyl glucose dioleate/hydroxystearate); methyl glucoside and isostearic acid ester (CTFA name: Methyl glucose isostearate); methyl glucoside and lauric acid ester (CTFA name: Methyl glucose laurate); the mixture of monoester and diester of methyl glucoside and isostearic acid (CTFA name: Methyl glucose sesqui-isostearate); the mixture of monoester and diester of methyl glucoside and stearic acid (CTFA name: Methyl glucose sesquistearate) and in particular the product marketed under the trade name Glucate SS by AMERCHOL, and mixtures thereof.

By way of oxyethylenated fatty acid and glucose or alkyl glucose ethers, mention may be made for example of oxyethylenated fatty acid and methyl glucose ethers, and in particular the polyethylene glycol ether of diester of methyl glucose and stearic acid containing approximately 20 moles of ethylene oxide (CTFA name: PEG-20 methyl glucose distearate) such as the product marketed under the trade name Glucam E-20 distearate by AMERCHOL; the polyethylene glycol ether of the mixture of monoester and diester of methyl glucose and stearic acid containing approximately 20 moles of ethylene oxide (CTFA name: PEG-20 methyl glucose sesquistearate) and in particular the product marketed under the trade name Glucamate SSE-20 by AMERCHOL and that marketed under the trade name Grillocose PSE-20 by GOLDSCHMIDT, and mixtures thereof.

By way of sucrose esters, mention may be made for example of sucrose palmito-stearate, sucrose stearate and sucrose mono laurate. By way of fatty alcohol ethers, mention may be made for example of ethers of polyethylene glycol and fatty alcohol comprising 8 to 30 carbon atoms, and particularly 10 to 22 carbon atoms, such as the ethers of polyethylene glycol and cetyl, stearyl, cetearyl (mixture of cetyl and stearyl alcohols) alcohols. Mention may be made for example of the ethers comprising from 1 to 200 and preferably from 2 to 100 oxyethylene groups, such as those having the CTFA name Ceteareth-20, Ceteareth-30, and mixtures thereof.

By way of sugar ethers, particular mention may be made of alkylpolyglucosides, and for example decylglucoside such as the product marketed under the trade name MYDOL 10 by Kao Chemicals, the product marketed under the trade name PLANTAREN 2000 by Henkel, and the product marketed under the trade name ORAMIX NS 10 by Seppic; caprylyl/capryl glucoside such as the product marketed under the trade name ORAMIX CG 110 by Seppic or under the trade name LUTENSOL GD 70 by BASF; laurylglucoside such as the products marketed under the trade names PLANTAREN 1200 N and PLANTACARE 1200 by Henkel; coco-glucoside such as the product marketed under the trade name PLANTACARE 818/UP by Henkel; cetostearyl glucoside optionally in a mixture with cetostearyl alcohol, marketed for example under the trade name MONTANOV 68 by Seppic, under the trade name TEGO-CARE CG90 by Goldschmidt and under the trade name EMULGADE KE3302 by Henkel, and arachidyl glucoside, for example in the form of the mixture of arachidic and behenic alcohols and arachidyl glucoside marketed under the trade name MONTANOV 202 by Seppic and any of the mixtures thereof.

According to one particular embodiment, the concentration of emulsifiers in the composition according to the invention ranges from 0.001 to 20%, preferably from 0.5 to 10% by mass with respect to the total mass of the composition.

According to one embodiment, the composition according to the invention may further comprise at least one preservative.

Advantageously, the preservative is a preservative routinely used in cosmetics. It may be chosen from the positive list contained in Annex VI of the Regulation (EC) No 1223/2009, which specifies the list of preservatives authorized in cosmetics. According to one alternative embodiment, the preservative(s) are present in the composition at a mass concentration of 0.001% to 10%, preferably from 0.1% to 2% with respect to the mass of the total composition.

The composition may further comprise a glycol chosen from propylene glycol, dipropylene glycol, butylene glycol, caprylyl glycol, glycerol and sorbitol, (poly)alkylene glycols and any of the mixtures thereof.

According to one embodiment, the mass concentrations of glycol range from 0.01% to 40%, by mass with respect to the total mass of said composition. Preferably, the mass concentrations of glycol range from 0.1% to 30%, and preferably from 5 to 20%, by mass with respect to the total mass of said composition.

Depending on the fluidity of the composition sought, one or a plurality of gelling agents, particularly hydrophilic, i.e. soluble or dispersible in water, may be incorporated in the composition. By way of hydrophilic gelling agents, mention may be made for example of optionally modified carboxyvinyl polymers, such as the products marketed under the trade names Carbopol (CTFA name: carbomer) and Pemulen (CTFA name: Acrylates/C 10-30 akyl acrylate crosspolymer) by Goodrich; polyacrylamides; 2-acrylamido 2-methylpropane sulfonic acid polymers and copolymers, optionally cross-linked and/or neutralized, such as poly(2-acrylamido-2-methylpropanesulfonic acid) marketed by Hoechst under the trade name “Hostacerin AMPS” (CTFA name: Ammonium Polyacryldimethyltauramide); cross-linked anionic acrylamide/AMPS copolymers, in the form of a W/O emulsion, such as those marketed under the name SEPIGEL 305 (C.T.F.A. name: Polyacrylamide/C13-14 lsoparaffin/Laureth-7) and under the name SIMULGEL 600 (C.T.F.A. name: Acrylamide/Sodium acryloyldimethyltaurate copolymer/Isohexadecane/Polysorbate 80) by SEPPIC; polysaccharide biopolymers such as xanthan gum, guar gum, alginates, modified celluloses; and mixtures thereof. The quantity of gelling agents is dependent on the end purpose. According to one embodiment, the quantity of gelling agents ranges from 0.001 to 10%, and for example from 0.1 to 5% by mass with respect to the total mass of the composition.

When the composition used according to the invention comprises an oil phase, the latter preferably contains at least one oil, particularly a cosmetic oil. It may further contain further fats.

By way of oils suitable for use in the composition according to the invention, mention may be made for example of:—hydrocarbon oils of animal origin, such as perhydrosqualene;—hydrocarbon oils of plant origin, such as fatty acid liquid triglycerides comprising 4 to 10 carbon atoms such as the triglycerides of heptanoic or octanoic acids or, for example sunflower, corn, soybean, pumpkin, grape seed, sesame, hazelnut, apricot kernel, macadamia, arara, sunflower, castor, avocado oil, caprylic/capric acid triglycerides such as those sold by Stearineries Dubois or those sold under the trade names Miglyol 810, 812 and 818 by Dynamit Nobel, jojoba oil, shea butter oil;—synthetic ethers and esters, particularly of fatty acids, such as the oils having the formulas R1COOR2 and R1OR2 wherein R1 represents the residue of a fatty acid comprising 8 to 29 carbon atoms, and R2 represents a hydrocarbon chain, optionally branched, containing 3 to 30 carbon atoms, such as for example Purcellin oil, isononyl isononanoate, isopropyl myristate, ethyl-2-hexyl palmitate, octyl-2-dodecyl stearate, octyl-2-dodecyl erucate, isostearyl isostearate; hydroxylated esters such as isostearyl lactate, octylhydroxystearate, octyldodecyl hydroxystearate, diisostearyl-malate, triisocetyl citrate, heptanoates, octanoates, decanoates of fatty alcohols; polyol esters, such as propylene glycol dioctanoate, neopentyl glycol diheptanoate and diethylene glycol diisononanoate; and pentaerythritol esters such as pentaerythrityl tetraisostearate;
linear or branched hydrocarbons, of mineral or synthetic origin, such as optionally volatile paraffin oils, and derivatives thereof, branched chain hydrocarbon oils comprising 10 to 20 carbon atoms such as isohexadecane, isododecane, isoparaffins and mixtures thereof, petroleum jelly, polydecenes, hydrogenated polyisobutene such as Parleam® oil;
natural or synthetic essential oils such as, for example, eucalyptus, lavandula, lavender, vetiver, litsea cubeba, lemon, sandalwood, rosemary, chamomile, savory, nutmeg, cinnamon, hyssop, caraway, orange, geraniol, cade and bergamot oils;
alcohols and fatty acids having 8 to 26 carbon atoms, such as cetyl alcohol or acid, stearyl alcohol, stearic acid, the mixture of cetyl alcohol and stearyl alcohol (cetylstearyl alcohol), octyl dodecanol, 2-butyloctanol, 2-hexyldecanol, 2-undecylpentadecanol, oleic acid or linoleic acid;
partially hydrocarbon and/or silicone fluorinated oils such as those described in the document JP-A-2-295912;
silicone oils such as polymethylsiloxanes (PDMS), optionally volatile with a linear or cyclic silicone chain, liquid or pasty at ambient temperature, particularly cyclopolydimethylsiloxanes (cyclomethicones) such as cyclohexasiloxane; polydimethylsiloxanes comprising alkyl, alkoxy or phenyl pendant or silicon chain-end groups, groups having 2 to 24 carbon atoms; phenyl silicones such as phenyltrimethicones, phenyldimethicones, phenyltrimethylsiloxydiphenyl-siloxanes, diphenyl-dimethicones, diphenylmethyldiphenyl trisiloxanes, 2-phenylethyltrimethyl-siloxysilicates, and polymethylphenylsiloxanes;—mixtures thereof.

The term “hydrocarbon oil” in the list of oils cited above denotes any oil comprising mostly carbon and hydrogen atoms, and optionally ester, ether, fluorinated, carboxylic acid and/or alcohol groups.

The other fats suitable for being present in the oil phase are for example fatty acids comprising 8 to 30 carbon atoms, such as stearic acid, lauric acid, palmitic acid and oleic acid; waxes such as lanolin, beeswax, Carnauba or Candellila wax, paraffin wax, lignite wax or microcrystalline waxes, ceresin or ozokerite, synthetic waxes such as polyethylene waxes, Fischer-Tropsch waxes; silicone resins such as trifluoromethyl-C1-4-alkyldimethicone and trifluoropropyldimethicone; and silicon elastomers such as the products marketed under the trade names “KSG” by Shin-Etsu, under the trade names “Trefil”, “BY29” or “EPSX” by Dow Corning or under the trade names “Gransil” by Grant Industries.

These fats may be chosen in varied ways by those skilled in the art in order to prepare a composition having the properties, for example in respect of consistency or texture, sought.

According to one alternative embodiment, the quantity of oil phase may range for example 0.01 to 60%, and for example from 5 to 50% by mass with respect to the total mass of the composition.

According to one alternative embodiment, the quantity of oil phase may range for example 10 to 40% by mass with respect to the total mass of the composition.

According to one embodiment, the composition further comprises a perfume.

According to one alternative embodiment, the quantity of perfumes may range for example 0.001 to 10%, and preferably from 0.01 to 5% by mass with respect to the total mass of the composition.

According to one particular embodiment, the composition is free from hydrotropic molecules such as for example nicotinamide (Vitamin B3), caffeine, sodium PCA, sodium salicylate, urea, hydroxyethyl urea and any of the mixtures thereof.

According to one particular embodiment, the composition comprises at least one hydrotropic molecule, such as for example nicotinamide (Vitamin B3), caffeine, sodium PCA, sodium salicylate, urea, hydroxyethyl urea and any of the mixtures thereof.

According to one aspect, the invention relates to a method for preparing a composition according to the invention.

According to one embodiment, the invention relates to a method for preparing a composition according to the invention, characterized in that it comprises steps for:

a) Mixing baicalin with at least water in order to obtain a mixture A containing baicalin; and

b) Optionally, adjusting the pH of mixture A to a pH greater than 6 by adding a base and/or an acid.

According to one embodiment, the invention relates to a method for preparing a composition according to the invention, characterized in that it comprises steps for:

a) Mixing baicalin with at least water in order to obtain a mixture A containing baicalin;

b) Increasing the pH of mixture A to a pH greater than 6 by adding a base; and

c) Lowering the pH to a pH of 5 to 6 by adding an acid, and optionally adjusted with a base.

A composition, preferably a cosmetic composition, is obtained, comprising at least one aqueous phase comprising baicalin, or an extract containing same, baicalin being solubilized and present at a mass concentration greater than 0.2%, or even 0.5% with respect to the total mass of the composition, said composition having a pH of 5 to 6. According to one embodiment, the invention relates to a method for preparing a composition according to the invention, characterized in that it comprises steps for:

a) Mixing baicalin with at least water in order to obtain a mixture A containing baicalin, baicalin being present at a concentration such that the mass concentration of baicalin is greater than or equal to 0.2%, or even 0.5%, with respect to the total mass of the composition;

b) Mixing, optionally in emulsification form, an oil phase with mixture A containing baicalin; and

c) Optionally, adjusting the pH to a pH of 5 to 6 by adding an acid and/or a base, in order to obtain a composition, preferably a cosmetic composition, comprising at least one aqueous phase comprising baicalin, or an extract containing same, baicalin being solubilized and present at a mass concentration greater than 0.2%, or even 0.5% with respect to the total mass of the composition, said composition having a pH of 5 to 6.

According to one embodiment, the solution containing baicalin is heated to a temperature between 60 and 80° C.

According to one alternative embodiment, the solution containing baicalin, optionally heated, may be added to an oil phase and/or a phase containing cosmetically acceptable excipients and/or cosmetically acceptable agents.

According to one alternative embodiment, the method according to the invention comprises steps for:

a) Mixing baicalin with at least water in order to obtain a mixture A containing baicalin, baicalin being present at a concentration such that the mass concentration of baicalin is greater than 0.2%, or even 0.5%, with respect to the total mass of the composition;

b) Increasing the pH of mixture A to a pH greater than 6 by adding a base;

c) Optionally, adjusting the pH to a pH of 5 to 6 by adding an acid and/or a base; and

b) Mixing, optionally in emulsification form, an oil phase with mixture A containing baicalin; and optionally

c) Optionally, adjusting the pH to a pH of 5 to 6 by adding an acid and/or a base; particularly so as to completely solubilize the baicalin.

Advantageously, if baicalin needles appear, the baicalin may then be completely solubilized by adding a base.

According to a further alternative embodiment, the method according to the invention comprises steps for:

a1) Mixing baicalin with at least water in order to obtain a mixture A containing baicalin, baicalin being present at a concentration such that the mass concentration of baicalin is greater than 0.2%, or even 0.5%, with respect to the total mass of the composition;

a2) Mixing, optionally in emulsification form, an oil phase with mixture A containing baicalin;

b) Increasing the pH of the mixture obtained to a pH greater than 7 by adding a base; and

c) Adjusting the pH to a pH of 5 to 6 by adding an acid, and optionally adjusted with a base.

According to one embodiment, the solution containing baicalin is heated to a temperature between 60 and 80° C. before being added to an oil phase, in order to form an emulsion.

According to one embodiment, the solution containing baicalin is heated to a temperature between 60 and 80° C. before being added to an oil phase containing an emulsifier, in order to form an emulsion.

According to one embodiment, the oil phase is heated to a temperature between 60 and 80° C. Advantageously, heating the oil phase enables complete dissolution of these components. In particular, it may be advantageous to heat the oil phase when it comprises UV filters, so as to completely solubilize the UV filters.

According to one embodiment, step c) is implemented at a temperature less than 35° C.

According to one alternative embodiment, the pH is adjusted prior to mixing the aqueous phase containing baicalin with another phase.

According to one alternative embodiment, the pH is adjusted after mixing the aqueous phase containing baicalin with another phase. At least one further, optional, step, routine for those skilled in the art, may be inserted into the preparation method.

When preparing a cosmetic composition, the method according to the present invention includes the addition of one or a plurality of excipients or active ingredient. This addition may be carried out in several goes, at various steps of the method.

According to one specific alternative embodiment, the method according to the invention comprises:
preparing an aqueous phase containing baicalin;
increasing the pH to a pH greater than 6, optionally heating to a temperature preferably between 60 and 80° C.,
preparing an oil phase, optionally containing UV filters, optionally heating the oil phase to a temperature between 60 and 80° C.,
mixing, for example in the form of emulsification, the aqueous phase containing baicalin and the oil phase optionally containing the UV filters,
adding one or a plurality of active ingredients and/or excipients when cooling the mixture of the aqueous phase and the oil phase, and
adjusting the pH from 5 to less than 6 at a temperature below 35° C.

According to one embodiment, the invention relates to the use of baicalin in a composition according to the invention as an antioxidant.

According to one aspect, the invention relates to the use of a concentration greater than or equal to 0.2% or even 0.5% of baicalin as an antioxidant in a cosmetic composition having a pH of 5 to 6, the concentration being expressed by mass with respect to the total mass of the composition. In one embodiment, said concentration is comprised between 0.25% and 10% of baicalin.

According to one embodiment, the invention relates to the use of the composition according to the invention for reducing the pigmentation induced by long UVA rays and/or by exposure to DUV.

According to one aspect, the invention relates to the use of the composition according to the invention for reducing the oxidation of the squalene contained in sebum induced by UV rays and the combination of UV rays and atmospheric pollution.

According to one aspect, the invention relates to a cosmetic and/or beauty treatment method comprising the topical application on the skin and/or lips and/or hair of a composition according to the invention or a composition susceptible to be obtained (or obtainable) by using a method according to the invention, for combating or preventing photo-induced premature aging of the skin and/or lips and/or hair.

According to one aspect, the invention relates to a cosmetic and/or beauty treatment method comprising the topical application on the skin and/or lips and/or hair of a composition according to the invention or a composition susceptible to be obtained (or obtainable) by using a method according to the invention, for protecting the skin and/or lips and/or hair against sun rays.

We will now give concrete examples illustrating the invention, but that are in no way restrictive.

All percentages given in the examples are given by mass, unless specified otherwise, and the temperature is ambient (20° C.) and expressed in degrees Celsius unless specified otherwise, and the pressure is atmospheric pressure, unless specified otherwise. In the examples, quantities of the ingredients of the compositions are given as a % by mass with respect to the total mass of the composition.

### EXAMPLES

### Examples 1 to 3

Photoprotective cosmetic compositions comprising baicalin at different concentrations.

The following oil-in-water emulsions were prepared:

| | | 1 | 2 | 3 |
|---|---|---|---|---|
| Phase | | (% by mass) | (% by mass) | (% by mass) |
| A1 | Water | qs 100 | Qs 100 | Qs 100 |
| | Caprylyl glycol | 0.3 | 0.3 | 0.3 |
| | Phenoxy-2 ethanol | 0.7 | 0.7 | 0.7 |
| | EDTA | 0.2 | 0.2 | 0.2 |
| | Baicalin (extract of | 1 (in extract) | 2.50 (in | 0.50 (in |
| | SCUTELLARIA | | extract) | extract) |
| | BAICALENSIS ROOT with | | | |
| | a baicalin content >95%) | | | |
| | Sodium hydroxide in | 0.027 in NaOH | up to pH 6.2-6.5 | up to pH 6.2-6.5 |
| | aqueous solution | up to pH 6.2-6.5 | | |
| A2 | Propylene Glycol | 7.5 | | |
| | Dipropylene glycol | 7.5 | | |
| | Xanthan gum | 0.25 | 0.25 | 0.25 |
| | Glycerin | | 15 | 15 |
| B | Dicaprylyl carbonate | 0.5 | 0.5 | 0.5 |
| | (BASF Cetiol CC) | | | |
| | Octocrylene | 7 | 7 | 7 |
| | ETHYLHEXYL | 5 | 5 | 5 |
| | SALICYLATE (SYMRISE | | | |
| | Neo Heliopan OS) | | | |
| | Homosalate | 5 | 5 | 5 |
| | Avobenzone | 3 | 3 | 3 |
| | Stearic acid | 1 | 1 | 1 |
| | Mixture (50/50 by mass) of | 3 | 3 | 3 |
| | glyceryl stearate and PEG- | | | |
| | 100 stearate (CRODA | | | |
| | Arlacel 165 FL) | | | |
| | Cetyl alcohol | 0.5 | 0.5 | 0.5 |
| | Dimethicone (visco 5 Cst) | 2 | 2 | 2 |
| C | Mixture of cross-linked poly | 1 | 1 | 1 |
| | dimethylsiloxane and poly | | | |
| | dimethylsiloxane (6 CST) | | | |
| | (24/76 by mass) (SHIN | | | |
| | ETSU KSG 16) | | | |
| D | CLARIANT AMMONIUM | 0.57 | 0.57 | 0.57 |
| | POLYACRYLOYLDIMETHYL | | | |
| | TAURATE HOSTACERIN | | | |
| | AMPS ® | | | |
| E | Silica spheres (5 μm) | 3 | 3 | 3 |
| F | Vitamin E | 1 | 1 | 1 |
| G | Perfume | 0.4 | 0.4 | 0.4 |
| H | Sodium hydroxide in | pH 5.5 | pH 5.5 | pH 5.5 |
| | aqueous solution at 50% | | | |
| | by mass or Citric acid qs | | | |

The total of the quantities of ingredients is equal to 100.0%. The acid and base are added in sufficient quantities to obtain the final pH sought.

The pH of the final composition is adjusted to pH 5.5 either with citric acid, or with sodium hydroxide.

The method for preparing the composition was as follows:

Phase A:
Mix the substances of phase A1, except for the base (sodium hydroxide). Stir using a magnetic stirrer, at ambient temperature.
Add the base (sodium hydroxide) until the baicalin is perfectly solubilized: phase A1 changes from an opalescent appearance to a transparent yellow solution appearance. Check under a polarized light microscope the disappearance of the needle-shaped baicalin crystals.
Prepare phase A2 by mixing the substances of phase A2 using a Rayneri type mixer for 5 minutes.
Add phase A2 to phase A1 and heat the mixture to 70° C.

Phase B:
Prepare phase B by mixing the materials of phase B and then heating to 75° C. until the UV filters have completely dissolved (translucent phase).

Emulsification:
Perform emulsification by stirring with the Moritz apparatus.
Slowly add phase B to phase A.
Stir for 5 minutes.
When the emulsion (consisting of phases B+A) reaches a temperature below 60° C., add substance C while stirring with the Moritz apparatus for 10 minutes.
Start to allow the emulsion to cool.
Add substance D while stirring with the Moritz apparatus for 10 minutes.
When the temperature reaches 45-50° C., add substances E, F and G and continue to stir with the Mortiz apparatus

Phase H:

Adjustment of pH of formula (to a temperature <35° C.)
Adjust the pH of the formula to 5.4-5.5 by adding some drops of base (sodium hydroxide solution) and/or of a 10% citric acid solution.
   Check the conformity of the formula using a polarized light microscope.
   If baicalin needles are present, then add one or a plurality of drops of base (sodium hydroxide) until the disappearance thereof.
      The cosmetic compositions obtained are presented in in the form of a yellow cream. The emulsions obtained are fine. The pH is 5.3. The pH is measured using a Seven Easy type pH-meter (Mettler Toledo, with temperature compensation) equipped with a pH Electrode combined with an InLab Solids Pro (Mettler Toledo) type integrated temperature probe. After having been calibrated, the pH-meter probe is introduced into the emulsion or aqueous phase.
      The compositions obtained are stable over time, at least 2 months at ambient temperature, for 2 months at 45° C. and at least 2 months at 4° C. This means that the properties thereof remain constant over time. The appearance of the composition does not change (or changes under acceptable conditions for the consumer); neither does the pH or viscosity thereof. The fineness of the emulsion remains constant over time. Baicalin does not recrystallize. Baicalin is not degraded (an analysis demonstrates that the mass concentration thereof remains constant).
      As such, the present invention makes it possible to provide a composition, particularly a cosmetic composition, that is both stable and comprising baicalin, or an extract containing same, at a mass concentration greater than or equal to 0.2% or 0.5%, while protecting baicalin from degradation or a recrystallization phenomenon.

### Examples 4 to 6

Cosmetic compositions comprising baicalin at different concentrations.

The following oil-in-water emulsions were prepared:

| | | | 5 | 6 |
|---|---|---|---|---|
| | | 4 | (% by | (% by |
| Phases | | (% by mass) | mass) | mass) |
| A | Water | qs 100 g | qs 100 g | qs 100 g |
| | Caprylyl glycol | 0.3 | 0.3 | 0.3 |
| | Phenoxy-2 ethanol | 0.7 | 0.7 | 0.7 |
| | EDTA | 0.15 | 0.15 | 0.15 |
| | Baicalin (extract of | 2.50 in | 1.00 in | 5.00 in |
| | SCUTELLARIA | extract | extract | extract |
| | BAICALENSIS ROOT with a | | | |
| | baicalin content >95%) | | | |
| | Potassium hydroxide in | 0.285 in KOH | 0.125 in | 0.5575 in |
| | aqueous solution at 50% by | up to pH 6.2-6.5 | KOH | KOH |
| | mass | | up to pH | up to pH |
| | | | 6.2-6.5 | 6.2-6.5 |
| | Propylene Glycol | 5.00 | 5.00 | 5.00 |
| | Dipropylene glycol | 5.00 | 5.00 | 5.00 |
| B | C12-C15 alkyl benzoate | 3.00 | 3.00 | 3.00 |
| | Isopropyl lauroyl sarcosinate | 0.80 | 0.80 | 0.80 |
| | (AJINOMOTO ELDEW SL- | | | |
| | 205 | | | |
| | Ethylhexyl palmitate | 3.00 | 3.00 | 3.00 |
| | Mixture (50/50 by mass) of | 2.20 | 2.20 | 2.20 |
| | glyceryl stearate and PEG- | | | |
| | 100 stearate (CRODA | | | |
| | Arlacel 165 FL) | | | |
| | Cetyl alcohol | 1 | 1 | 1 |
| | Tocopherol | 1 | 1 | 1 |
| C | Dimethicone (viscosity at | 2.50 | 2.50 | 2.50 |
| | 25° C. 100 Cst) | | | |
| | Xanthan gum | 0.30 | 0.30 | 0.30 |
| | AMMONIUM | 0.60 | 0.60 | 0.60 |
| | POLYACRYLOYLDIMETHYL | | | |
| | TAURATE (CLARIANT | | | |
| | HOSTACERIN AMPS ®) | | | |
| D | Water | 20.00 | 20.00 | 20.00 |
| | Potassium hydroxide | qs | qs | qs |
| E | MICA (and) TITANIUM | 0.02 | 0.02 | 0.02 |
| | DIOXIDE (and) RED 28 | | | |
| | LAKE (and) TIN OXIDE | | | |
| F | Mixture of cross-linked poly | 7.50 | 7.50 | 7.50 |
| | dimethylsiloxane and poly | | | |
| | dimethylsiloxane (6 tcST at | | | |
| | 25° C.) (24/76 by mass) (SHIN | | | |
| | ETSU KSG 16) | | | |
| | Mixture of DIMETHICONE | 0.50 | 0.50 | 0.50 |
| | and DIMETHICONOL (DOW | | | |
| | CORNING XIAMETER PMX- | | | |
| | 1503 FLUID) | | | |
| G | METHYLSILANOL/SILICATE | 0.50 | 0.50 | 0.50 |
| | CROSSPOLYMER | | | |
| | (TAKEMOTO NLK 506) | | | |
| H | Denatured ethanol | 3.00 | 3.00 | 3.00 |
| I | Potassium hydroxide in | pH 5.5 | pH 5.5 | pH 5.5 |
| | aqueous solution at 50% by | | | |
| | mass or Citric acid qs | | | |

These compositions are stables; they may be applied onto facial skin as an anti-age treatment and to reduce the pigmentation induced by UV, particularly long UVA rays and DUV.

### Example 7

The exemplified composition 3 has been prepared as mentioned in examples 1-3 above, with several content (mass concentration) of baïcalin, between 0.3% and 1%, with a pH of 5.5 according to the invention or of 6.2 (which is not encompassed by the present invention).

The coloration of the compositions has then been evaluated.

### Evaluation Protocol:

The color of the compositions has been evaluated after the preparation of thin films on contrast cards. The compositions were deposited in a 2.2 cm diameter circle and were leveled to obtain a reproducible thickness deposit.
The colorimetric measurements were performed with a spectro-colorimeter Minolta CM2600D on two points of the film. The test is reproduced twice, leading to 4 measures by tested composition.
The results are expressed in the system (L*, a*, b*) in which L* represents luminance, a* represents the red-green axis (−a*=green, +a*=red) and b* represents the yellow-blue axis (−b*=blue, +b*=yellow). Therefore, a* and b* express the shade of the tested composition. The hue angle h* allows defining an acceptance criterion for the color. The value of h* is obtained from the a* and b* value: h*=arctan (b*/a*).
The h* value should be comprised between 110 and 135.
The results are shown in Table 1 here-below.

| % | | | | | | | | Color |
|---|---|---|---|---|---|---|---|---|
| Baïcalin | pH | Test | L* | a* | b* | C* | h* | acceptance |
| 0.3 | 5.5 | C | 92 | −4 | 8.5 | 9.4 | 115.2 | YES |
| 0.3 | 6.2 | D | 92 | −2.6 | 10.8 | 11.1 | 103.5 | NO |
| 0.5 | 5.5 | E | 92 | −6.2 | 11.8 | 13.3 | 117.7 | YES |
| 0.5 | 6.2 | F | 92 | −4 | 19.1 | 19.5 | 101.8 | NO |
| 1 | 5.5 | G | 92 | −7.5 | 14 | 15.9 | 118.2 | YES |
| 1 | 6.2 | H | 91 | −4.9 | 30 | 30.4 | 99.3 | NO |

These results show that with a mass concentration of baicalin superior to 0.25% and a pH comprised between 5 and 6, the color of the composition has an orange tint less pronounced than compositions having a pH of 6.2.

1. A cosmetic composition comprising at least one aqueous phase comprising baicalin, or an extract containing same, baicalin being solubilized and present at a mass concentration between 0.25% and 10% with respect to the total mass of the composition, said composition having a pH of 5 to 6 and said composition being an emulsion.

2. The cosmetic composition according to claim 1, which comprises an acid and a base.

3. The cosmetic composition according to claim 1, wherein baicalin, or an extract containing same, is present at a mass concentration ranging between 0.25% to 8%.

4. The cosmetic composition according to claim 1, wherein the pH of the composition is comprised between 5.1 and 5.9.

5. The cosmetic composition according to claim 1, which further comprises at least one vitamin chosen from vitamin A (retinol), vitamin E (tocopherol), vitamin C (ascorbic acid), vitamin CG (ascorbyl glucoside), vitamin B5 (panthenol), derivatives of these vitamins (particularly esters) and any of the mixtures thereof.

6. The cosmetic composition according to claim 1, which further comprises at least one active agent.

7. The cosmetic composition according to claim 1, which further comprises at least one UV filter chosen from organic hydrophilic UV filters, organic lipophilic UV filters, insoluble organic UV filters, mineral filters or any of the mixtures thereof.

8. The cosmetic composition according to claim 1, which it further comprises at least one emulsifier.

9. A method for preparing a composition according to claim 1, wherein it comprises steps for:
a) Mixing baicalin with at least water in order to obtain a mixture A containing baicalin; and
b) Optionally, adjusting the pH of mixture A to a pH greater than 6 by adding a base and/or an acid.

10. A method for preparing a composition according to claim 1, wherein it comprises steps for:
a) Mixing baicalin with at least water in order to obtain a mixture A containing baicalin;
b) Increasing the pH of mixture A to a pH greater than 6 by adding a base; and
c) Lowering the pH to a pH of 5 to 6 by adding an acid, and optionally adjusted with a base.

11. A cosmetic composition comprising as an antioxidant baicalin at a concentration between 0.25% and 10% in the cosmetic composition, wherein the cosmetic composition has a pH of 5 to 6, the concentration being expressed by mass with respect to the total mass of the composition.

12. The cosmetic composition according to claim 1, for reducing the pigmentation induced by long UVA rays and/or by DUV exposure.

13. A cosmetic and/or beauty treatment method comprising the topical application on the skin and/or lips and/or hair of a composition as defined in claim 1, for combating or preventing photo-induced premature aging of the skin and/or lips and/or hair.

14. A cosmetic and/or beauty treatment method comprising the topical application on the skin and/or lips and/or hair of a composition as defined in claim 1, for protecting the skin and/or lips and/or hair against sun rays.

15. The cosmetic composition according to claim 2, wherein baicalin, or an extract containing same, is present at a mass concentration ranging between 0.25% to 8%.

16. The cosmetic composition according to claim 2, wherein the pH of the composition is comprised between 5.1 and 5.9.

17. The cosmetic composition according to claim 3, wherein the pH of the composition is comprised between 5.1 and 5.9.

18. The cosmetic composition according to claim 2, which further comprises at least one vitamin chosen from vitamin A (retinol), vitamin E (tocopherol), vitamin C (ascorbic acid), vitamin CG (ascorbyl glucoside), vitamin B5 (panthenol), derivatives of these vitamins (particularly esters) and any of the mixtures thereof.

19. The cosmetic composition according to claim 3, which further comprises at least one vitamin chosen from vitamin A (retinol), vitamin E (tocopherol), vitamin C (ascorbic acid), vitamin CG (ascorbyl glucoside), vitamin B5 (panthenol), derivatives of these vitamins (particularly esters) and any of the mixtures thereof.

20. The cosmetic composition according to claim 4, which further comprises at least one vitamin chosen from vitamin A (retinol), vitamin E (tocopherol), vitamin C (ascorbic acid), vitamin CG (ascorbyl glucoside), vitamin B5 (panthenol), derivatives of these vitamins (particularly esters) and any of the mixtures thereof.
